(19) United States
(12) Patent Application Publication
(10) Pub. No.: US 2011/0275834 A1
(43) Pub. Date: Nov. 10, 2011

(54) POLYMORPH OF STRONTIUM RANELATE AND A PROCESS FOR ITS PREPARATION

(75) Inventors: Murali Krishna Prasad Divi, Hyderabad (IN); Mysore Aswatha Narayana Rao, Hyderabad (IN); Shaik Nowshuddin, Hyderabad (IN)

(73) Assignee: DIVI'S LABORATORIES, LIMITED, Hyderabad (IN)

(21) Appl. No.: 12/983,398

(22) Filed: Jan. 3, 2011

(30) Foreign Application Priority Data
May 6, 2010 (IN) 1267/CHE/2010

(51) Int. Cl. C07D 333/38

(52) U.S. Cl. 549/61

(57) ABSTRACT

This invention discloses Strontium Ranelate polymorph, designated as Form A, having a characteristic powdered x-ray diffraction pattern and infrared spectrum with a water content of about 1.5 to 2.5% and a process for its preparation.

### CROSS-REFERENCE TO RELATED APPLICATIONS

This application claims priority from India Application Serial No. 1267/CHE/2010, filed May 6, 2010, entitled Polymorph of Strontium Ranelate and a Process for Its Preparation, which application is assigned to the same assignee as this application and whose disclosure is incorporated by reference herein.

### FIELD OF INVENTION

The present invention discloses a novel polymorph of strontium ranelate having a water content of 1.5 to 2.5% and a process for its preparation.

### BACKGROUND OF THE INVENTION

Strontium ranelate is the distrontium salt of 5-[bis(carboxymethyl)amino]-3-carboxymethyl-4-cyano-2-thiophenecarboxylic acid and is represented by Formula I.

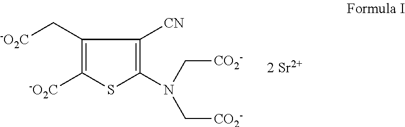

Strontium ranelate is used in treating osteoporosis and other bone diseases. It increases bone formation and also reduces bone resorption, resulting in a rebalance of bone turnover in favor of bone formation.

### DESCRIPTION OF THE PRIOR ART

Strontium salts such as lactate, gluconate, and carbonate were known to be useful in the treatment of bone diseases such as osteoporosis (Proc. Staff Meetings Mayo Clinic, 1960, 35, 105-111; Can. Med. Assoc. 1981, 125, 703-712; Trace Subst. Environ. Health 1984, 18, 3-23). However the strontium salt of ranelate was found to have a distinct advantage over other salts because of its superior bioavailability (U.S. Pat. No. 5,128,367).

The preparation and the therapeutic uses of strontium ranelate are described in U.S. Pat. No. 5,128,367. The '367 patent describes three methods to prepare strontium ranelate. In the first method, the ethyl tetraester of Formula II was hydrolyzed to get free acid and the acid was dissolved in water and treated with strontium hydroxide.

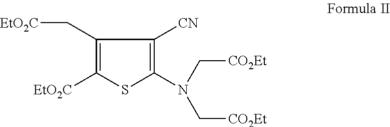

The crystals of strontium ranelate were obtained as an octahydrate. In the second method, the tetraester was hydrolyzed to the sodium salt of the acid and its aqueous solution was treated with strontium chloride. The strontium ranelate crystallized out as the octahydrate. In the third method, the tetraester was directly hydrolyzed with strontium hydroxide in an aqueous solution to obtain the octahydrate form of strontium ranelate.

U.S. Pat. No. 7,459,568B2 describes an alpha crystalline form of strontium ranelate and a process for its preparation. The process consists of dissolving strontium ranelate octahydrate in water, followed by refluxing the solution and cooling to obtain the alpha crystalline form of strontium ranelate. It has a water content of 22-24%, which accounts for its octahydrate nature. The alpha crystalline form has been further characterized by powdered X-ray diffraction.

WO 2007/020527 A2 describes a process for preparing strontium ranelate octahydrate from the tetra ester through use of a lithium base.

Chinese patent, CN 101108845 (A) describes a method to prepare strontium ranelate heptahydrate where the tetra ester was hydrolyzed using an aqueous alcoholic solution of sodium hydroxide and the sodium salt treated with strontium chloride. The heptahydrate obtained by this method had a 19 to 20.4% water content.

Strontium ranelate is available in the market as “PROTELOS”, which is a 2 gram (g) sachet containing granules of strontium ranelate. The granules are to be added to water, stirred and taken orally as a suspension. The daily recommended dose is 2 g as a single dose.

The marketed “PROTELOS” product contains strontium ranelate as its octahydrate. High moisture content of strontium ranelate, to the extent of about 22% indicates that only about 78% of the drug is available per unit weight. Since the dose is large, the bulk of the formulation is very large which is undesirable for many reasons.

Those in the art would like to have strontium ranelate with a lower moisture content so that the size of the formulation can be minimized. There is thus a need for a compact form of strontium ranelate with a low moisture content, which could be used in pharmaceutical formulations.

### SUMMARY OF THE INVENTION

The present invention provides a novel form of strontium ranelate having a much reduced water content, of about 1.5 to 2.5%, allowing for a smaller size formulation.

This novel strontium ranelate is a stable crystalline polymorph having a characteristic powdered X-ray diffraction pattern as shown in FIG.-1.

The invention also provides a process for the preparation of strontium ranelate having about 1.5 to 2.5% water content and having characteristic powdered X-ray diffraction pattern as shown in FIG.-1.

### DETAILED DESCRIPTION OF THE INVENTION

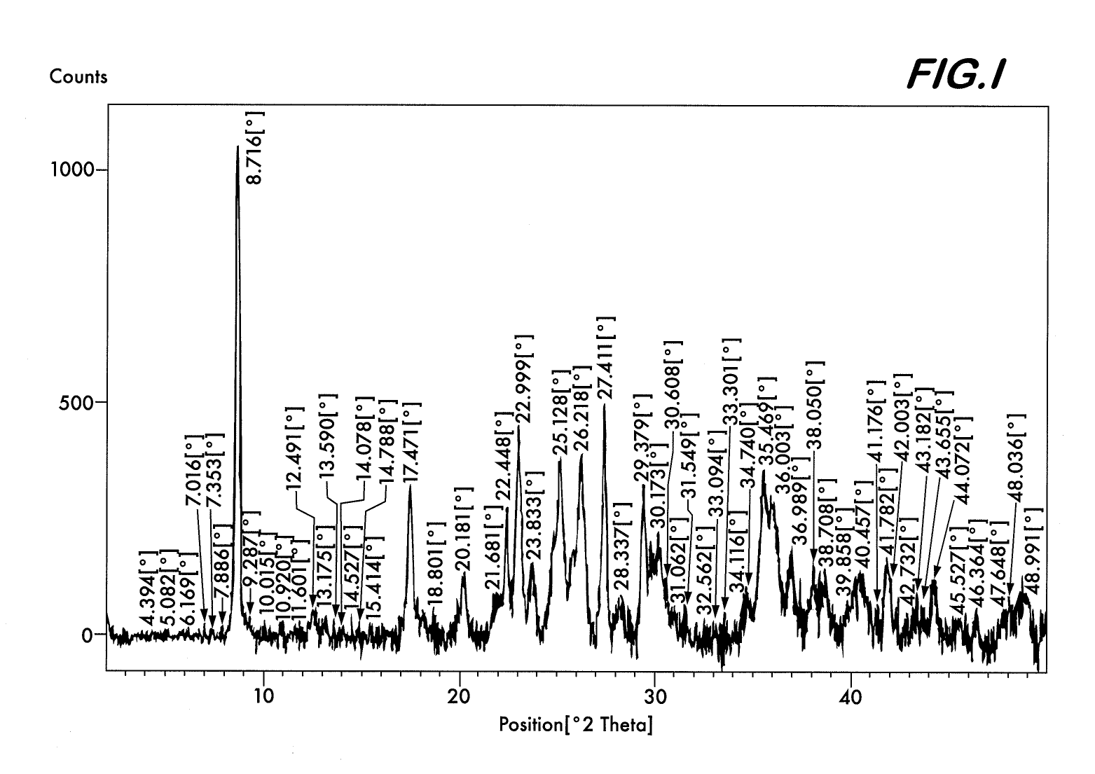

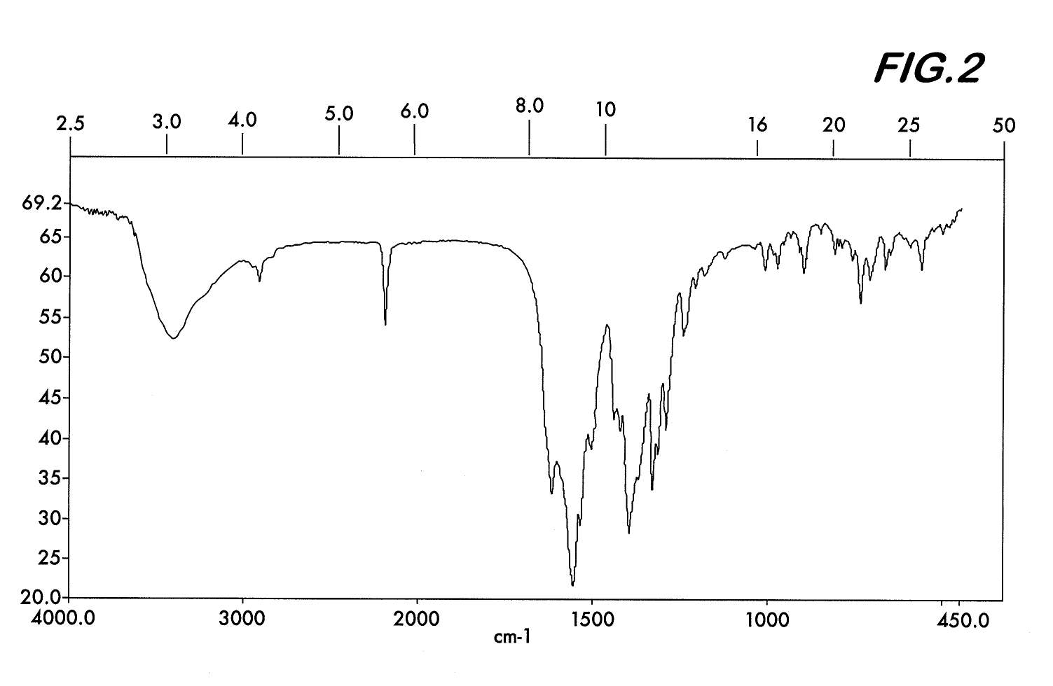

In the present invention, we describe a new crystalline form of strontium ranelate with about 1.5-2.5% water content. This new polymorph has been designated as Form A. It is stable and can be characterized by any five or more peaks in the powdered X-ray diffraction pattern given in FIG. 1 including, but not limited to, 8.71, 17.48, 22.37, 23.03, 25.13, 26.20, 27.34, 29.43, and 35.44 degrees 2-theta and infrared absorption spectrum as given in FIG. 2 including, but not limited to 3413.6, 2209,8, 1622.9, 1558.1, 1398.8, 1295.7, and 1246.9 cm⁻¹.

The present invention also describes a process for the preparation of Form A which comprises azeotropic drying of strontium ranelate octahydrate by refluxing in a suitable solvent or antisolvent using Dean-Stark or any other apparatus to trap the released water.

The preferred solvent to carry out the present invention is toluene. However, other solvents with a high boiling point can also be used. After refluxing, the reaction mixture is cooled, filtered and further dried under vacuum to obtain a product with a moisture content of about 1.5 to 2.5%. This product shows the characteristic peaks in powdered X-ray diffraction and infrared spectrum. The starting material, strontium ranelate octahydrate used in the process can be prepared by a process described in U.S. Pat. No. 5,128,367 or by any other method known to one skilled in the art.

The main advantage of strontium ranelate form A is its very low water content (about 1.5 to 2.5%) compared to 22% in the octahydrate or 19-20% in heptahydrate forms. The formulations prepared using Form A will have a higher API content on a weight by weight basis. Because of its low water content, the stability of the formulation will also improve to a significant extent.

### EXAMPLE

Strontium ranelate octahydrate (20 g) was suspended in 250 ml of toluene and refluxed in a Dean-Stark apparatus for 8 hours and collected water removed. The reaction mixture was cooled to room temperature and filtered. The filtered solid was further dried under vacuum to obtain 15.5 g of the product (98% yield, 99.6% purity by HPLC) with a moisture content of 1.8% w/w. It showed the characteristic powdered X-ray diffraction pattern as in FIG. 1 and IR pattern as in FIG. 2.

Without further elaboration the foregoing will so fully illustrate our invention that others may, by applying current or future knowledge, adopt the same for use under various conditions of service.

We claim :

1. Strontium ranelate of formula 1 having a water content of about 1.5 to 2.5% w/w.

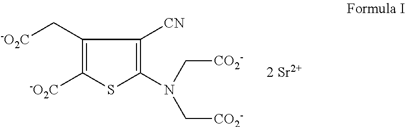

2. A crystalline polymorph of the strontium ranelate of claim 1 that exhibits a powdered X-ray diffraction pattern having characteristic peaks expressed in degrees 2 theta at approximately 8.71, 17.48, 22.37, 23.03, 25.13, 26.20, 27.34, 29.43, and 35.44.

3. A crystalline polymorph of the strontium ranelate of claim 1 that exhibits an infrared absorption spectrum in KBr having characteristic absorption bands approximately at, 3413.6, 2209,8, 1622.9, 1558.1, 1398.8, 1295.7, and 1246.9 cm⁻¹.

4. A process for the preparation of the crystalline polymorph of the strontium ranelate as in claim 1 which comprises suspending strontium ranelate or its hydrate in a suitable solvent and refluxing the suspension to remove water by azeotropic distillation.

5. The process of claim 4 wherein the solvent is toluene.

6. A process for the preparation of the crystalline polymorph of the strontium ranelate as in claim 2 which comprises suspending strontium ranelate or its hydrate in a suitable solvent and refluxing the suspension to remove water by azeotropic distillation.

7. The process of claim 6 wherein the solvent is toluene.

8. A process for the preparation of the crystalline polymorph of the strontium ranelate as in claim 3 which comprises suspending strontium ranelate or its hydrate in a suitable solvent and refluxing the suspension to remove water by azeotropic distillation.

9. The process of claim 8 wherein the solvent used is toluene.

10. A crystalline polymorph of the strontium ranelate of claim 1 that exhibits a powdered X-ray diffraction pattern having characteristic peaks expressed in degrees 2 theta at approximately 8.71, 17.48, 22.37, 23.03, 25.13, 26.20, 27.34, 29.43, and 35.4. and an infrared absorption spectrum in KBr having characteristic absorption bands approximately at, 3413.6, 2209,8, 1622.9, 1558.1, 1398.8, 1295.7, and 1246.9 cm⁻¹.
